(19) United States
(12) Patent Application Publication
(10) Pub. No.: US 2021/0113363 A1
(43) Pub. Date: Apr. 22, 2021

(54) MENSTRUAL CUP WITH STRUCTURAL FEATURES

(71) Applicants: Courtney Rose Evans, Murray, UT (US); Lexie Rose, Murray, UT (US)

(72) Inventors: Courtney Rose Evans, Murray, UT (US); Lexie Rose, Murray, UT (US)

(21) Appl. No.: 17/076,780

(22) Filed: Oct. 21, 2020

Related U.S. Application Data
(60) Provisional application No. 62/924,164, filed on Oct. 21, 2019.

(51) Int. Cl. A61F 5/455; A61F 5/44

(52) U.S. Cl. CPC A61F 5/4553; A61F 5/4404

(57) ABSTRACT

A menstrual cup includes a cup comprising a circumferential wall and an opening, wherein the opening leads to a cavity within the wall. The menstrual cup further includes a stem at a base of the cup, the stem protruding from the cup at a side opposite the opening, and a plurality of ridges positioned on an exterior surface of the cup proximate the base of the cup.

### CROSS-REFERENCES TO RELATED APPLICATIONS

This application claims the benefit of U.S. Provisional Patent Application No. 62,924,164 entitled “MENSTRUAL CUP WITH STRUCTURAL FEATURES” and filed on Oct. 21, 2019 for Courtney Rose Evans et al., which is incorporated herein by reference.

### FIELD

Embodiments of methods and apparatuses are described for feminine products, specifically menstrual cups.

### BACKGROUND

A menstrual cup is a feminine product used to catch and collect menstrual flow rather than absorbing it. They are used without an applicator and made such that the cup portion is deformable by the user prior to insertion of the device into the vagina. Once inserted, the cup should reform allowing the perimeter of the cup opening to rest against the vaginal wall forming a seal. In this way, menstrual flow is captured. The problem with many menstrual cups is that they do not consistently reform or reshape properly to cause a sufficient seal after insertion. This can cause unwanted leakage and may require annoying reapplications. Additionally, many existing menstrual cups do not include a way to twist the menstrual cup after insertion, which in many cases is necessary to assure or confirm a proper seal between the menstrual cup and the vaginal wall. Further, many existing menstrual cups are not comfortably functional. They are often too rigid in their structure or utilize certain materials that while providing structure, may be irritating to the vaginal wall.

What is needed is a menstrual cup that more consistently reforms or reshapes within the vagina after being deformed upon insertion. It would further be an improvement over existing menstrual cups to have a cup that facilitates twisting the inserted cup. It would also be an improvement over existing menstrual cups that provide proper functionality while also providing comfort. Such a menstrual cup is described herein.

### SUMMARY

The subject matter of the present application has been developed in response to the present state of the art, and in particular, in response to the problems and disadvantages associated with conventional menstrual cups that have not yet been fully solved by currently available techniques. Accordingly, the subject matter of the present application has been developed to provide embodiments that overcome at least some of the shortcomings of prior art techniques.

Disclosed herein is a menstrual cup. The menstrual cup includes a cup comprising a circumferential wall and an opening, wherein the opening leads to a cavity within the wall. The menstrual cup further includes a stem at a base of the cup, the stem protruding from the cup at a side opposite the opening, and a plurality of ridges positioned on an exterior surface of the cup proximate the base of the cup. The preceding subject matter of this paragraph characterizes example 1 of the present disclosure.

The stem protrudes from the cup in a direction orthogonal to a plane of the opening. The preceding subject matter of this paragraph characterizes example 2 of the present disclosure, wherein example 2 also includes the subject matter according to example 1, above.

The cup comprises a lip at the opening and the lip is an annular protrusion protruding circumferentially around the opening on an interior surface of the cup. The preceding subject matter of this paragraph characterizes example 3 of the present disclosure, wherein example 3 also includes the subject matter according to any one of examples 1-2, above.

The cup comprises a lip at the opening, and the lip comprises a curved surface. The preceding subject matter of this paragraph characterizes example 4 of the present disclosure, wherein example 4 also includes the subject matter according to any one of examples 1-3, above.

The cup comprises a lip at the opening, the lip is an annular protrusion protruding circumferentially around the opening on an interior surface of the cup, and the lip comprises an interior curvilinear angled surface and an exterior curvilinear angled surface. The preceding subject matter of this paragraph characterizes example 5 of the present disclosure, wherein example 5 also includes the subject matter according to any one of examples 1-4, above.

The stem comprises an oblong cross-sectional shape with at least one planar surface. The preceding subject matter of this paragraph characterizes example 6 of the present disclosure, wherein example 6 also includes the subject matter according to any one of examples 1-5, above.

The stem comprises grooves circumventing the stem. The preceding subject matter of this paragraph characterizes example 7 of the present disclosure, wherein example 7 also includes the subject matter according to any one of examples 1-6, above.

The plurality of ridges positioned on the exterior surface of the cup are linear. The preceding subject matter of this paragraph characterizes example 8 of the present disclosure, wherein example 8 also includes the subject matter according to any one of examples 1-7, above.

The plurality of ridges positioned on the exterior surface of the cup are wavy. The preceding subject matter of this paragraph characterizes example 9 of the present disclosure, wherein example 9 also includes the subject matter according to any one of examples 1-8, above.

The cup comprises at least one hole in the wall of the cup. The preceding subject matter of this paragraph characterizes example 10 of the present disclosure, wherein example 10 also includes the subject matter according to any one of examples 1-9, above.

The cup comprises a lip at the opening, the lip is an annular protrusion protruding circumferentially around the opening on an interior surface of the cup, an internal surface of the cup comprises at least one internal rib, and the at least one internal rib extends from the lip to a base of the cavity. The preceding subject matter of this paragraph characterizes example 11 of the present disclosure, wherein example 11 also includes the subject matter according to any one of examples 1-10, above.

Disclosed herein is a menstrual cup. The menstrual cup includes a cup comprising a circumferential wall and an opening, wherein the opening leads to a cavity within the wall. The menstrual cup further includes a stem at a base of the cup, the stem protruding from the cup at a side opposite the opening, and a plurality of ridges positioned on an exterior surface of the cup proximate the base of the cup. The cup comprises a lip at the opening and the lip is an annular protrusion protruding circumferentially around the opening on an interior surface of the cup. The preceding subject matter of this paragraph characterizes example 12 of the present disclosure.

The lip comprises a curved surface. The preceding subject matter of this paragraph characterizes example 13 of the present disclosure, wherein example 13 also includes the subject matter according to any one of examples 1-12, above.

The lip comprises an interior curvilinear angled surface and an exterior curvilinear angled surface, and the interior curvilinear angled surface is convex, and the exterior curvilinear angled surface is concave. The preceding subject matter of this paragraph characterizes example 14 of the present disclosure, wherein example 14 also includes the subject matter according to any one of examples 1-13, above.

The stem protrudes from the cup in a direction orthogonal to a plane of the opening. The preceding subject matter of this paragraph characterizes example 15 of the present disclosure, wherein example 15 also includes the subject matter according to any one of examples 1-14, above.

The cup comprises at least one hole in the wall of the cup. The preceding subject matter of this paragraph characterizes example 16 of the present disclosure, wherein example 16 also includes the subject matter according to any one of examples 1-15, above.

The plurality of ridges positioned on the exterior surface of the cup are wavy, and wherein the plurality of ridges is circumferentially equally spaced around the wall of the cup. The preceding subject matter of this paragraph characterizes example 17 of the present disclosure, wherein example 17 also includes the subject matter according to any one of examples 1-16, above.

The stem comprises grooves circumventing the stem. The preceding subject matter of this paragraph characterizes example 18 of the present disclosure, wherein example 18 also includes the subject matter according to any one of examples 1-17, above.

The plurality of ridges positioned on the exterior surface of the cup are linear. The preceding subject matter of this paragraph characterizes example 19 of the present disclosure, wherein example 19 also includes the subject matter according to any one of examples 1-18, above.

Disclosed herein is a menstrual cup. The menstrual cup includes a cup comprising a circumferential wall and an opening, wherein the opening leads to a cavity within the wall. The menstrual cup further includes a stem at a base of the cup, the stem protruding from the cup at a side opposite the opening, and a plurality of ridges positioned on an exterior surface of the cup proximate the base of the cup. The cup comprises a lip at the opening and the lip is an annular protrusion protruding circumferentially around the opening on an interior surface of the cup. The cup comprises a lip at the opening, wherein the lip is an annular protrusion protruding circumferentially around the opening on an interior surface of the cup, and wherein the lip comprises a curved surface. The stem protrudes from the cup in a direction orthogonal to a plane of the opening. stem comprises grooves circumventing the stem, and an internal surface of the cup comprises at least one internal rib, and the at least one internal rib extends from the lip to a base of the cavity. The preceding subject matter of this paragraph characterizes example 20 of the present disclosure.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 depicts a perspective view of a menstrual cup in accordance with one or more embodiments of the present invention;

FIG. 2 depicts a side plan view of a menstrual cup with a cup portion facing down and a stem portion facing up in accordance with one or more embodiments of the present invention;

FIG. 2A depicts a representation of a range of orthogonality relative to a baseline in accordance with one or more embodiments of the present invention;

FIG. 3 depicts a top plan view of the menstrual cup shown in FIG. 2 and oriented as shown in FIG. 2 in accordance with one or more embodiments of the present inventions; and

FIG. 4 depicts a bottom plan view of the menstrual cup shown in FIG. 2 and oriented as shown in FIG. 2 in accordance with one or more embodiments of the present invention;

FIG. 5 depicts a side plan view of the menstrual cup shown in FIG. 2 rotated 90 degrees about central axis defined by cross-section A-A in accordance with one or more embodiments of the present invention;

FIG. 6 depicts a side plan cutaway view of FIG. 5 along cross-section A-A in accordance with one or more embodiments of the present invention;

FIG. 7 depicts a side cutaway view of a menstrual cup in accordance with one or more embodiments of the present invention;

FIG. 8 depicts a side cutaway view of a menstrual cup in accordance with one or more embodiments of the present invention;

FIG. 9 depicts a top view of a menstrual cup in accordance with one or more embodiments of the present invention; and

FIG. 10 depicts a bottom view of a menstrual cup in accordance with one or more embodiments, of the present invention.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Embodiments of a menstrual cup according to the present invention are generally described herein. Although described and illustrated in the appended figures in a particular way, the parts and features of the menstrual up could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale.

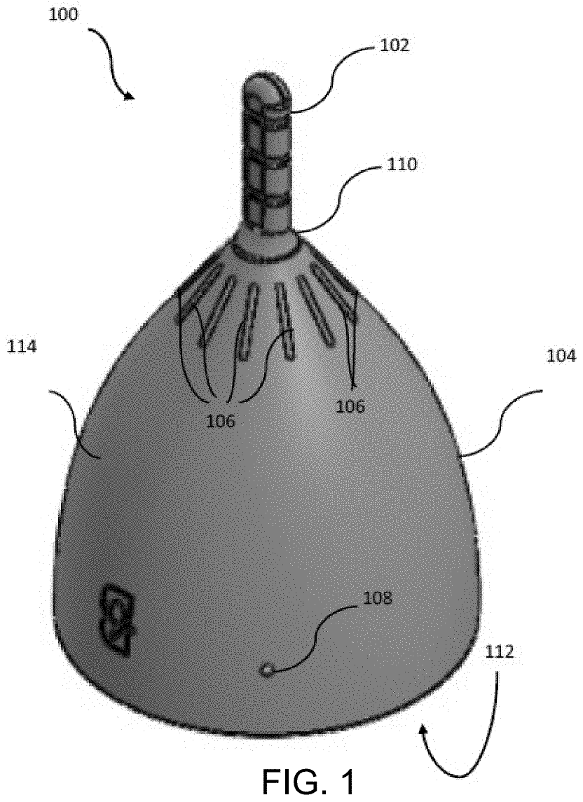

Turning now to FIG. 1, a perspective view of a menstrual cup 100 is shown. The cup may have a stem 102, a cup portion 104, one or more ridges 106, and one or more holes 108. The cup portion 104 may be configured with a closed stem side 110 and an opening 112 positioned opposite the stem side 110. The opening 112 allows the menstrual cup 100 to hold fluid when positioned above the stem 102 with the opening 112 facing upward. The menstrual cup 100 is contoured such that a diameter (not shown) of the stem side 110 of the cup portion 104 is smaller than a diameter (not shown) of the opening 112 of the cup portion 104. In one embodiment, the cup portion is bell shaped. The stem 102 is configured to facilitate insertion, removal and orientation of the menstrual cup 100.

The ridges 106 may be positioned on exterior surface 114 of the cup portion 104. In one embodiment, the ridges 106 are positioned closer to the stem side 110 of the cup portion 104 than the opening 112 of the cup portion 104. In another embodiment, the ridges 106 are positioned adjacent the stem side 110 of the cup portion 104. The ridges 106 may be configured to facilitate gripping of the menstrual cup 100, and particularly the cup portion 104 of the menstrual cup, by a user after the menstrual cup 100 has been inserted into the vagina.

One or more of the holes 108 may be formed within the cup portion 104 of the menstrual cup 100. In one embodiment, one or more of the holes 108 are positioned within the cup portion 104 closer to the opening 112 of the cup portion 104 than the stem side 100 of the cup portion 104. In another embodiment one or more of the holes 108 are positioned adjacent the opening 112 of the cup portion. The holes 108 may be sized, positioned or otherwise configured to help introduce air into an interior (not shown) of the cup portion 104 to help the cup portion fully open after it has been folded, pinched or otherwise deformed upon insertion. The holes 108 may also be sized, positioned or otherwise configured to help the cup portion 104 of the menstrual cup 100 get a good seal against the vaginal wall. This can occur as a result of pinching or otherwise deforming the cup portion 104 upon insertion, because some of the air is forced out through the holes 106 thereby creating a slight suction. The holes 108 may also be sized, positioned or otherwise configured to release a suction upon removal of the cup 100 from the vagina. As the cup portion 104 is deformed upon removal, air may be allowed into the interior be up portion 104 breaking the suction and allowing the menstrual cup 100 to be removed with increased comfort.

The stem 102, cup portion 104 and ridges 106 may be made of a pliable material. In one embodiment, the stem 102, cup portion 104 and ridges 106 are made of rubber. In other embodiment, the stem 102, cup portion 104 and ridges 106 are made of a silicone material. In yet another embodiment, the stem 102, cup portion 104 and ridges 106 are made of platinum silicone. Other embodiments utilize different materials such as lower-grade silicones or TPE. It will be appreciated that a variety of medical grade silicones and rubbers may be used to form the menstrual cup 100 or the individual parts thereof.

The stem 102 and cup portion 104 may be formed as one unitary piece or may be formed separately and joined together by ways know in the art. In embodiments where one or more of the stem 102, cup portion 104 and ridges 106 are formed separately and then joined together, one or more of the stem 102, cup portion 104 and ridges 106 may be made of separate material. The individual parts of the menstrual cup 100 may made by a number of ways know in the art to form pliable materials, including without limitation, blow molding or compression molding, or similar techniques. In one embodiment, at least an exterior surface 114 of the cup portion 104 has a smooth finish. In another embodiment, the exterior surface 114 of the cup portion 104 has a satin finish. Different embodiments might be implemented with combinations of different finishes.

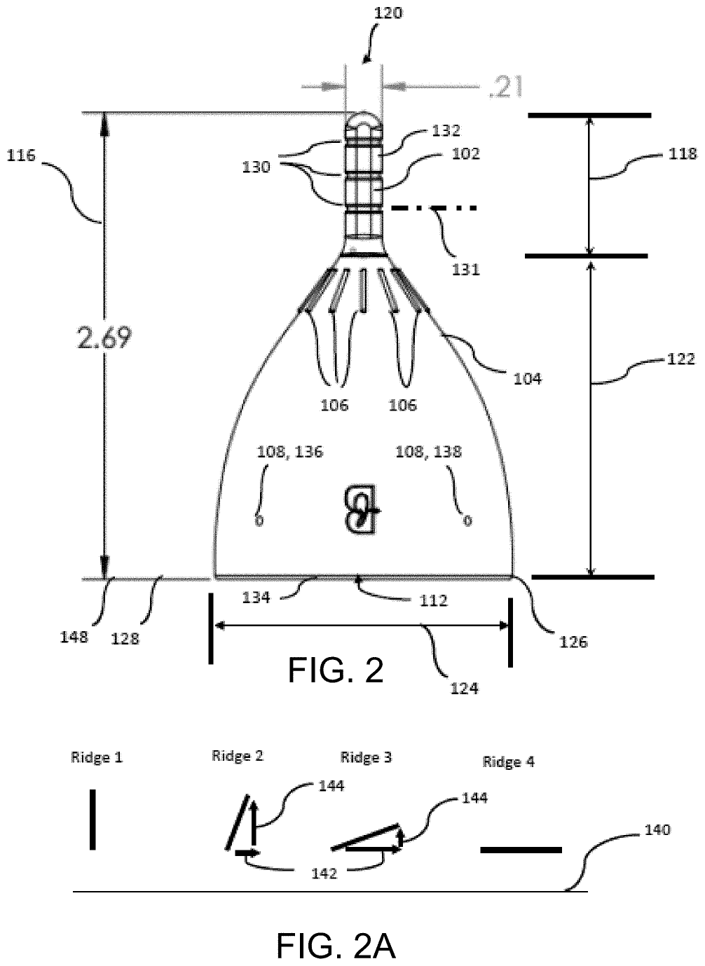

Turning now to FIG. 2, a side plan view of the menstrual cup 100 is shown with the cup portion 104 positioned beneath the stem 102. It will be appreciated that this positioning is for ease of describing embodiments of the invention and that orientation of the menstrual cup 100 with the cup portion 104 above the stem 102 is required for proper function. A length 116 of the menstrual cup 100 in one embodiment may be between about 2 inches and about 3 inches. In one embodiment, the length 116 is between about 2.2 inches and about 2.8 inches. In another embodiment, the length 116 is about 2.5 inches. The stem 102 may have a length 118 of between about 0.5 inches and about 3 inches. In another embodiment the stem length 118 may be between about 1 inch and about 2 inches. The stem 102 may have a width 120 of between about 0.05 inches and 0.5 inches. In another embodiment, the stem 102 may have a width 120 of between about 0.1 and 0.3 inches. The cup portion 104 may have a length 122 of between about 0.5 inches and about 2.5 inches. In another embodiment the cup portion 104 may have a length 122 of between about 1.0 inch and 1.7 inches. The opening 112 may have width 124 between about 0.5 inches and about 3 inches. In another embodiment, the opening 112 has a width between about 1 inch and about 2 inches. In yet another embodiment, the opening 112 is substantially circular and the width 124 is a diameter. The diameter in this embodiment may be between about 1.4 inches and 1.8 inches.

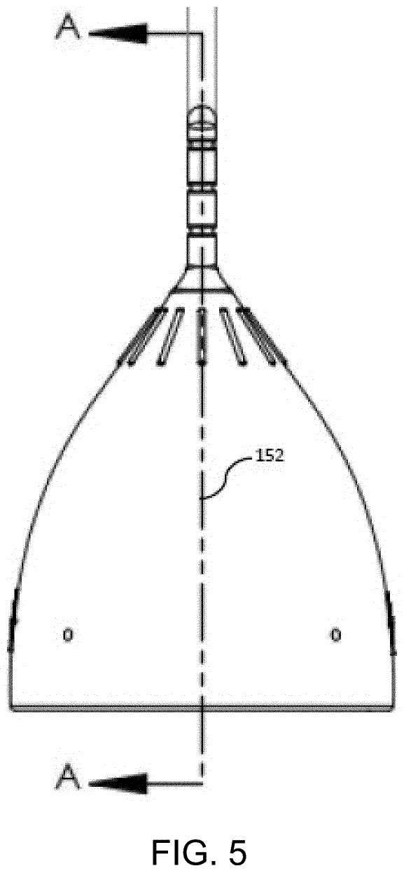

The opening 112 (or cross section 126 of the cup portion 104 that is orthogonal to a central axis (see FIG. 5) of the menstrual cup 100) may define a plane 128. The stem 102 may be configured with one or more grooves 130. In one embodiment, one or more grooves are annular formed within an exterior surface 132 of the stem. In another embodiment, three or more grooves 130 are formed equidistantly along the stem at intervals of between about 0.1 inches and about 0.4 inches. In yet another embodiment one or more grooves are spaced at varying distances from one another, with at least one pair of grooves being about 0.2 inches apart. The grooves 130 may be configured to facilitate a user gripping the step 102. A portion of at least one of the grooves 130 may lie a plane 131 that is parallel with plane 128. The grooves 130 may also act as a score on the surface 132 of the stem 102 to facilitate a straight cut through the stem 102, thus shortening the length 118 of the stem 102. In this configuration, the length 116 of the menstrual cup 100 may be customized by the user.

One or more holes 108 may be spaced about an outer or external surface 114 of the cup portion 104 towards the opening 112. In one embodiment, there are 2 holes 108 spaced about a perimeter of the external surface 114 of the cup portion 104. In another embodiment, there are 3 holes 108 spaced about a perimeter of the external surface 114 of the cup portion 104. In yet another embodiment, 4 holes 108 are equally spaced about a perimeter of the externa surface 114 of the cup portion 104. At least one hole 108 may be positioned between about 0.1 inch and 0.5 inches from an edge 134 of the opening 112. In another embodiment, at least one hole 108 may be positioned about 0.3 inches from an edge 134 of an opening 112. In one embodiment, a first hole 108, 136 is positioned at the same distance from an edge 134 of the opening 112 than a second hole 108, 138. In another embodiment, a first hole 108, 136 is positioned at a different distance from an edge 134 of the opening 112 than a second hole 108, 138. At least one hole 108 may be formed through a surface 114 of the cup portion 104 that engages a vaginal after insertion. In one embodiment, all of the one or more holes 108 are positioned through a surface 114 of the cup portion 104 that engages a vaginal well when inserted.

One or more ridges 106 may be positioned about a perimeter of the exterior surface 114 of the cup portion 104 of the menstrual cup 100. The ridges 106 may be the same or different lengths than one or more other ridges 106. The ridges 106 may be spaced at equal distances about a perimeter of the exterior surface 114 or at varying distances. The ridges 106 may be straight or curved. Indeed, the ridges 106 may have two ends (not shown), or 3 or more ends in a branched configuration, or no ends in a circular or other continuous configuration. In one embodiment, a portion of at least one ridge 106 is between about 0.05 inches and 0.5 inches from the stem side 110 of the cup portion 104. In another embodiment, a portion of at least one ridge 106 is between about 0.1 and 0.3 inches from the stem side 110 of the cup portion 104. It will be appreciated that the size and positioning of the ridges 106 about the cup portion 104 facilitates gripping the cup portion 104 when the menstrual cup 100 is inserted within the vagina.

Referring now to FIG. 2A, a series of four straight ridges 106 are numbered and shown in different positions relative to a base line 140. Ridge 1 is substantially perpendicular or orthogonal to the baseline. Ridge 2 is angled at about 80 degrees relative to the baseline and thus contains a parallel vector 142 and an orthogonal vector 144. Because the amount of the orthogonal vector 144 is greater than the parallel vector 142, Ridge 2 can be said to be more orthogonal than parallel to the baseline. Ridge 3 is angled at about 20 degrees relative to the baseline and thus contains a parallel vector 142 and an orthogonal vector 144, where the parallel vector 142 is greater than the orthogonal vector 144. Because the amount of the orthogonal vector 144 is less than the parallel vector 142, Ridge 3 can be said to be more parallel than orthogonal to the baseline. Ridge 4 is parallel to the baseline.

A straight ridge can be described as having an orthogonal element, or at least some orthogonality if it has an orthogonal vector 144 or a vector orthogonal to a baseline 140 of any size. Curved ridges 106 can have orthogonal elements or orthogonality if at any point along the curve, the normal vector, or in other words a vector perpendicular to the curve is not parallel to the baseline 140. Indeed, as used herein, every curved ridge 106 would have an orthogonal element or orthogonality. In fact, as used herein, the only ridge 106 without an orthogonal element or at least some orthogonality would be a ridge 106 in the shape of a straight line that is parallel to the baseline 140. Curved ridges 106 may be more orthogonal than parallel to a baseline 140 if the curve contains a tangent at any part of the curve that has an orthogonal element or component that is greater than a parallel component or element. By way of non-limiting example, and by way a shallow sinusoidal curve relative to a horizontal line may not have any tangents to any part of the curve that form acute angles with the horizontal line of less than 45 degrees. These may be said to be more parallel than orthogonal relative to the horizontal line. In contrast, a deep sinusoidal curve relative to a horizontal line may have any tangents to any part of the curve that form acute angles with the horizontal line of more than 45 degrees. Ridges of this shape may be said to have a portion that is orthogonal than parallel relative to the horizontal line, even though the curve has a whole may travel or extend horizontally.

To make the above-discussion applicable to the three dimensional menstrual cup 100 of the present invention, a two dimensional plan view representation of the three dimensional object can be used (see FIGS. 2-6) to establish the baseline and a view presented such that the contour of the three-dimensional object does not affect the angle of a ridge in a two-dimensional view.

Returning again to FIG. 2, at least one of the ridges 106 has a portion that does not lie in plane or circle parallel to plane 128. Stated another way, at least one of the ridges 106 has some orthogonality relative to a baseline defined as the plane 128 represented by the line 148. In another embodiment, at least one ridge 106 is more orthogonal than parallel to plane 128 represented by line 148.

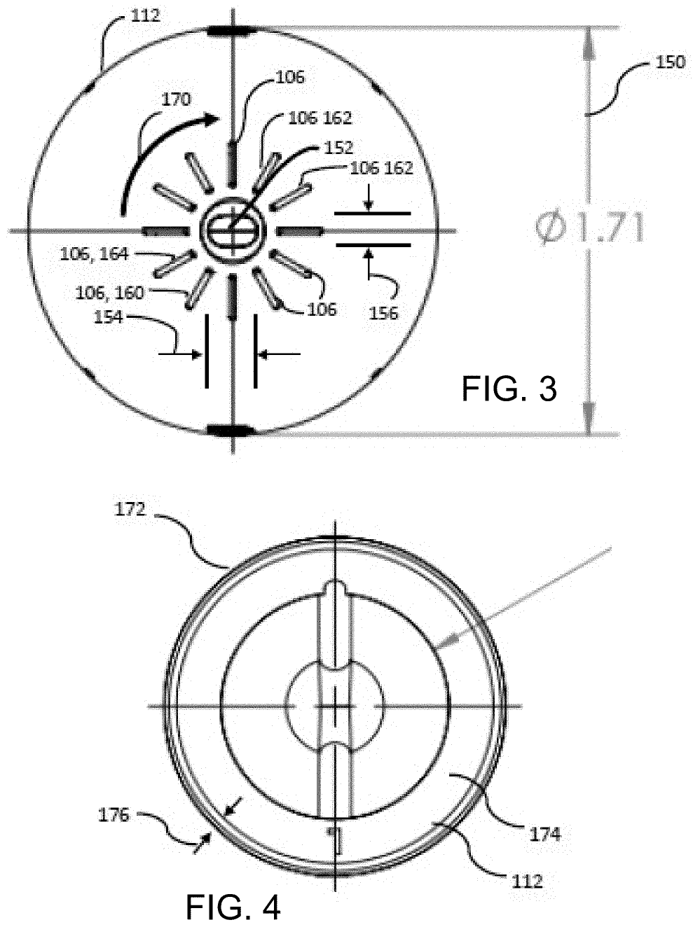

Referring now to FIG. 3, a top plan view of the menstrual cup 100 is shown. In this embodiment, the opening 112 is circular having a diameter 150 between about 1 inch and 3 inches. In another embodiment, the diameter 150 is between about 1.4 inches and about 1.8 inches. The menstrual cup 100 has a central axis 152. The ridges 106 may be positioned about the axis 152 on the exterior surface 114 of the cup portion 104 toward the step side 110 of the cup portion 104 of the menstrual cup 100. Figure three shows a cross section of the stem 102. The stem 102 cross section may have a length 154 and a width 156. In one embodiment, the length 154 is between about between about 0.1 inches and about 0.3 inches and the width 156 is between about 0.05 inches and about 0.2 inches. In embodiments where the cross-section length 154 is greater than the cross-section width 156, the stem 102 has a planar configuration which provides flat surface areas 158 for easier user gripping.

In one embodiment, at least one ridge 106, 160 is positioned across a center point defined by the axis 152 on an opposite surface from another ridge 106, 162. Stated another way, for substantially circular cup portions 104, at least one ridge 106, 164 is positioned on a surface of the cup portion 104 about 180 degrees around the perimeter of the cup portion 104 from another ridge 106, 166.

At least one ridge 106 of the menstrual cup 100 includes a portion 168 that is parallel to a baseline defined by any radial extending from the central axis 152. In another embodiment, the menstrual cup 100 includes a ridge 106 with portions on opposing sides of the menstrual cup 100 that are parallel to a radial extending from the central axis 152. In the embodiment shown in FIG. 3, each of the ridges 106 are parallel to a radial extending from the central axis 152. In one embodiment, the menstrual cup 100 includes at least one ridge 106, not shown that is not perpendicular to a radial extending from the central axis. In another embodiment, the menstrual cup 100 includes at least one ridge that is less perpendicular or has less orthogonality than it is parallel to a radial extending from the central axis 152. In another embodiment the menstrual cup 100 includes at least one ridge that is less parallel than perpendicular or orthogonal to a radial extending from the central axis 152.

It will be appreciated that it may be advantageous for a user to rotate the menstrual cup 104 within the vagina to help it seal against the vaginal wall. In one embodiment, one or more of the ridges 106 are positioned on the surface 114 of the cup portion 104 to facilitate this rotation. In one embodiment, the ridges are positioned such that an edge or surface or portion of the ridges is not in line with or parallel with or concentric with a direction of rotation 170 about the central axis 152. It will further be appreciated that a ridge that is essentially concentric with the rotation 170 of the menstrual cup 104 may not have sufficient surface area to allow enough friction to twist the menstrual cup 100 within the vagina. With an edge or surface or portion of a ridge 106 that is not concentric with the rotation motion 170, there is at least a portion that the thumb and/or finger of a user can push against to create a rotational force to overcome the force of friction between the menstrual cup 100 and the vaginal wall. The more perpendicular the ridge (or a portion edge or surface thereof) is to the direction of twist rotation 170 about the central axis, the more edge or surface or portion of the ridge 106 there is to push against to create a rotational force that is sufficient.

Turning now to FIG. 4, a bottom plan view of the menstrual cup 100 of FIG. 2 is shown. In the embodiment shown, the menstrual cup 100 has an opening 112 defined be a circular perimeter 172 about the central axis 152. In alternative embodiments, the opening 112 may be of a different shape. The menstrual cup 100 may include and annular piece 174 about the perimeter 172 of the cup portion 104. The annular piece 174 may provide spill protection as will be discussed in further detail below. In one embodiment, the cup portion 104 of the menstrual cup 104 has a uniform thickness 176. In other embodiments, the thickness 176 may vary.

Referring now to FIG. 5, a menstrual cup 100 according to the present invention is shown. The cutaway line A-A bisects the cup along a central axis 152.

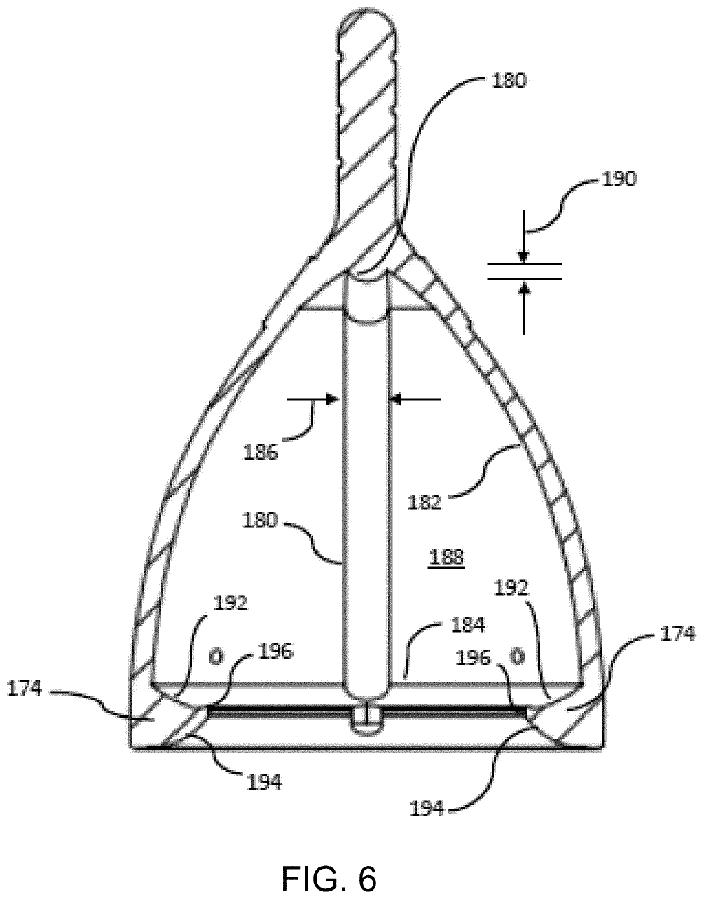

FIG. 6 shows a cutaway of the menstrual cup 100 along line A-A. The cutaway view reveals an internal rib 180. In one embodiment, the rib 180 extends along an interior surface 182 of the cup portion 104 from one side 184 of the opening 112 in the cup portion to an interior surface of the stem side 110 of the cup portion 104 and back to an opposing side (not shown) of the opening 112. The shape of the rib 180 substantially matches a cross-section of the interior of the cup portion 104 through a central axis. In one embodiment, the rib 180 is separately configured and then added to the interior of the cup portion 104. In another embodiment, the rib 180 is configured as part of a unitary menstrual cup 100, as shown in FIG. 6. In one embodiment, the rib 180 has a pair of opposing arms, that when attached to the interior surface, or molded as part of the interior surface of the cup portion 104, form a thicker portion along a cross section of the menstrual cup 104. In one embodiment, the rib 180 has a width 186 between about 0.1 inches and about 0.3 inches. In another embodiment, the rib 180 has a width 186 between about 0.15 inches and about 0.22 inches. In one embodiment, the rib 180 extends into the interior 188 of the cup portion 104 by a distance 190 of between about 0.001 inches and about 0.4 inches.

It will be appreciated by those of skill in the art that a thicker deformable material will reform to its original shape after deformation with more force or strength than the same thinner deformable material similarly deformed. Accordingly, with the rib 180 attached to, or made part of, a wall of the cup portion 104 will help the deformed wall of the cup portion 104 reform more consistently than a similar wall without a rib 180. In one embodiment, a plurality of ribs 180 are configured within the interior 190 of the cup portion 104 to facilitate reformation of the cup portion 104 after it has been deformed upon insertion into the vagina. In one embodiment the cup portion 104 has a thicker portion extending from an exterior surface of the cup portion 104. In this embodiment, the thicker portion may not extend all the way to the edge of an opening of the cup portion so as to not interfere with the seal of the outer surface of the cup portion 104 against the vaginal wall. In one embodiment, the rib 180 has a pair of arms (not shown) that are connected to each other at the step side 110 of the cup portion 104 and attached to at least one of the interior surface 182 of the cup or an exterior surface (not shown). It will be appreciated that as the arms of the rib 180 are pinched together to deform the cup portion 104 to facilitate insertion, the arms will push against each other during reformation, thus helping to reform the cup portion 104.

The menstrual cup 100 may have an annular piece 174 configured with an interior curvilinear angled surface 192. The interior curvilinear angled surface 192 may extend from an interior surface 182 of the cup portion toward the interior 188 at the opening 112. In this configuration, any captured menstrual flow traveling up the interior wall due to motion of the cup will be redirected by the interior curvilinear angled surface 192 toward the interior 188 of the cup or to an area where it will be more likely to be recaptured in the cup, rather than exit the cup.

The annular piece 174 may also include an exterior curvilinear angled surface 194 extend from a point near the opening 112 down into the cup, when the cup is oriented for use. It will be appreciated that the annular exterior curvilinear angled surface 194 creates a funneling feature to guide menstrual flow into the cup portion 104 of the cup 100. On or more of the interior curvilinear angled surface 192 and the exterior curvilinear angled surface 194 may be curved to facilitate retention and funneling respectively. In one embodiment the interior curvilinear angled surface 912 and exterior curvilinear angled surface 194 meet at a point 196 within the cup portion 104 of the menstrual cup 100.

In one embodiment the annular piece 174 may be formed and then connected at an interior surface of the cup portion 104 near the opening 112. In another embodiment, the annular piece 174 may be formed as an integral part of the cup portion 104. Parts described herein as being attached or connected may be accomplished by any number of ways known in the art, including without limitation, welding, gluing, fusing, melting and the like. Similarly, parts or described herein as being made, created, generated, formed, or with similar descriptions, may be done in a variety of ways known in the art.

Accordingly, embodiments of a menstrual cup have been described herein that are pliable, and that have features configured to help reform more efficiently and accurately within the vagina after deformation upon insertion into the vagina. Additionally, embodiments of a menstrual cup have been described herein that are better suited to allow twisting of the cup while inserted into the vagina.

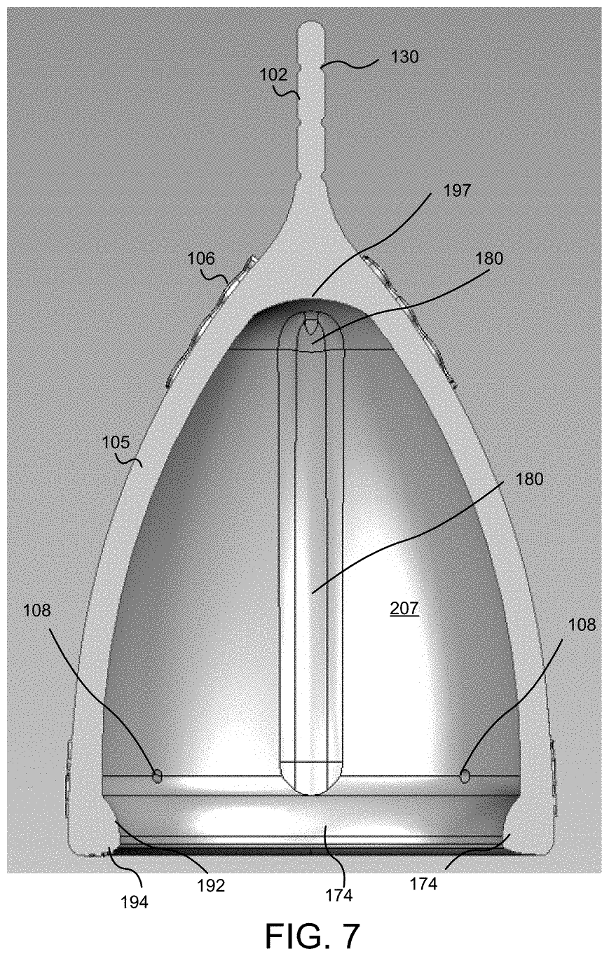

Referring now to FIG. 7, another embodiment of a menstrual cup 100 is shown. The menstrual cup 100 includes many similar features to other embodiments described herein, many of which features are not described in detail again for the sake of brevity. The menstrual cup 100 includes a cup including a circumferential wall 105, and an opening 112. The opening 112 leads to a cavity 207 within the circumferential wall 105 with a base 197 of the cavity opposite the opening 112.

The menstrual cup 100 includes a stem 102 at a base 197 of the cup. The stem 102 is located at a side opposite the opening 112 and protrudes out the exterior of the cup. The menstrual cup 100 includes a plurality of ridges 106 which are positioned on an exterior surface 114 of the wall 105 near the base 197 of the cup. The ridges 106 are wavy protrusions that extend out of the exterior surface 114. In some embodiments, the ridges 106 include other shapes and configurations. In some embodiments, the ridges 106 are equally spaced circumferentially around the cup extending for the stem to a distance down the wall 105 of the cup.

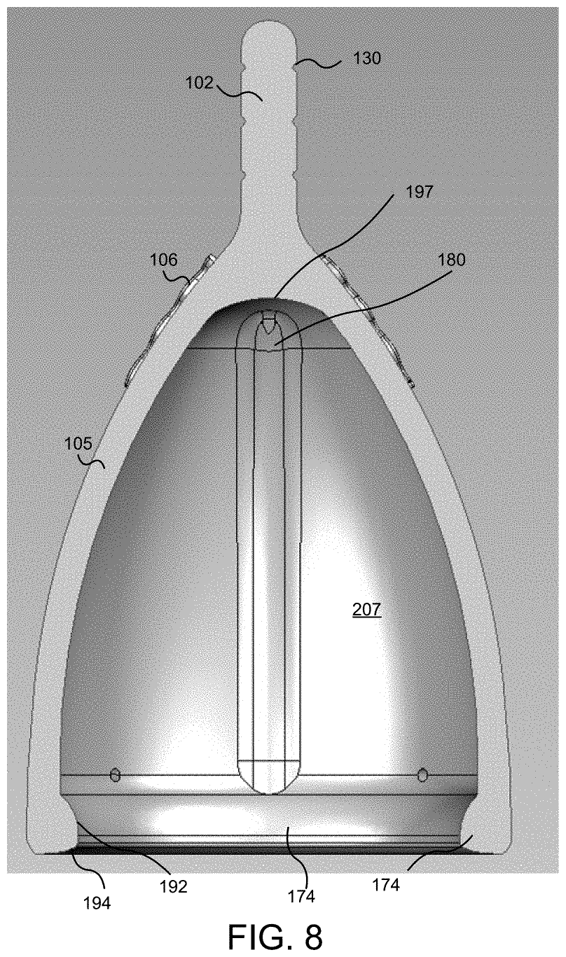

In some embodiments, the stem 102 protrudes from the cup in a direction orthogonal to the plane of the opening 112. Referring now to both FIGS. 7 and 8, the stem 102 includes a width and length which differ. The views of FIGS. 7 and 8 are orthogonal to each other which depicts and contrasts the narrow width of the stem 102 in FIG. 7 with the longer length of the stem 102 in FIG. 8.

In some embodiments, the stem 102 includes one or more annular grooves 130. The annular grooves 130 circumferentially navigate around the stem in a plane orthogonal to the direction that the stem 102 protrudes from the cup. The size and number of grooves may vary in embodiments. The grooves 130 and the planar surface of the stem 102 allow for ease of control of the menstrual cup 100 during any manual manipulation of the menstrual cup 100.

In some embodiments, the menstrual cup 100 includes a plurality of holes 108 that extend through the wall 105 of the cup. The number and size of the holes 108 may vary in embodiments. In addition, the location of the holes 108 may also vary in embodiments. In some embodiments, the holes 108 are located proximate the opening 112. In some embodiments, the holes 108 are spaced apart equally across the wall 105 of the cup.

In some embodiments, the menstrual cup 100 includes a lip 174, referred to earlier as an annular piece, which is an annular protrusion that protrudes circumferentially around the opening 112 on an interior surface of the cup. In some embodiments, the lip 174 includes a curved surface. In some embodiments, the lip 174 includes an interior curvilinear angled surface 192 and an exterior curvilinear angled surface 194, the exterior being located nearer to the opening and the interior being located on the interior of the cup.

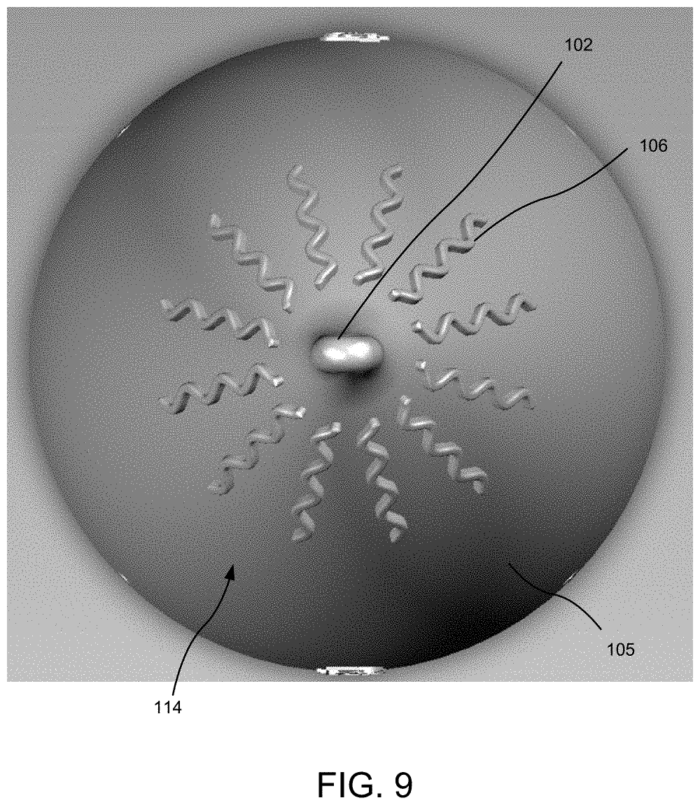

Referring now to FIG. 9, a top view of the menstrual cup 100 is shown. FIG. 9 more clearly depicts the cross section of the stem 102 and the shape and position of the ridges 106 which are wavy in the illustrated embodiment. The ridges 106 extend out and in a direction away from the stem 102 and are protrusions that include a wave-like shape.

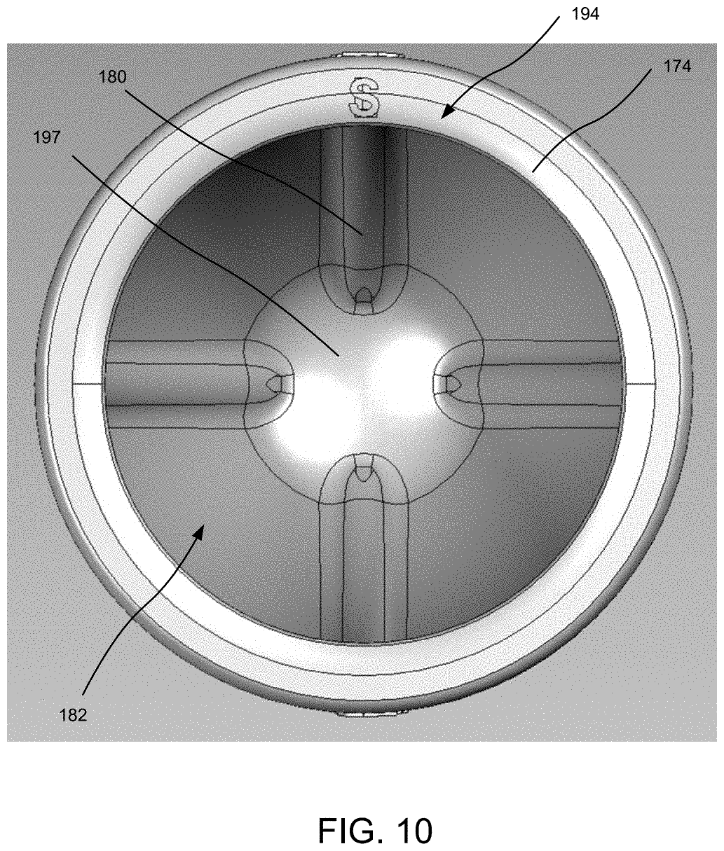

Referring now to FIG. 10, a bottom view of the menstrual cup 100 is shown. FIG. 10 more clearly depicts the base 197 of the cup and the internal or interior ribs 180. In the illustrated embodiment, there are four ribs 180 spaced equidistant around the interior surface 182 of the cup and extending from the base 197 to near or at the lip 174 of cup.

FIG. 10 further shows the lip 174 and the exterior curvilinear angled surface 194 that are circumferentially surrounding the opening 112 of the cup. The shape and size of the lip 174 may vary as shown by the various embodiments described herein. The shape and size may be optimized for the individual use of the menstrual cup 100. The annular protrusion allows for the cup to retain contents within the cup upon extraction of the device.

Although the foregoing disclosure provides many specifics, these should not be construed as limiting the scope of any of the ensuing claims. Other embodiments may be devised which do not depart from the scopes of the claims. Features from different embodiments may be employed in combination. The scope of each claim is, therefore, indicated and limited only by its plain language and the full scope of available legal equivalents to its elements.

Reference throughout this specification to features, advantages, or similar language does not imply that all of the features and advantages that may be realized with the subject matter of the present disclosure should be or are in any single embodiment. Rather, language referring to the features and advantages is understood to mean that a specific feature, advantage, or characteristic described in connection with an embodiment is included in at least one embodiment of the present disclosure. Thus, discussion of the features and advantages, and similar language, throughout this specification may, but do not necessarily, refer to the same embodiment.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The present subject matter may be embodied in other specific forms without departing from its spirit or essential characteristics. The described embodiments are to be considered in all respects only as illustrative and not restrictive.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity

What is claimed is:

1. A menstrual cup, comprising:
a cup comprising a circumferential wall and an opening, wherein the opening leads to a cavity within the wall;
a stem at a base of the cup, the stem protruding from the cup at a side opposite the opening; and
a plurality of ridges positioned on an exterior surface of the cup proximate the base of the cup.

2. The menstrual cup of claim 1, wherein the stem protrudes from the cup in a direction orthogonal to a plane of the opening.

3. The menstrual cup of claim 1, wherein:
the cup comprises a lip at the opening, and
the lip is an annular protrusion protruding circumferentially around the opening on an interior surface of the cup.

4. The menstrual cup of claim 3, wherein:
the cup comprises a lip at the opening, and
the lip comprises a curved surface.

5. The menstrual cup of claim 1 wherein:
the cup comprises a lip at the opening,
the lip is an annular protrusion protruding circumferentially around the opening on an interior surface of the cup, and
the lip comprises an interior curvilinear angled surface and an exterior curvilinear angled surface.

6. The menstrual cup of claim 1, wherein the stem comprises an oblong cross-sectional shape with at least one planar surface.

7. The menstrual cup of claim 1, wherein the stem comprises grooves circumventing the stem.

8. The menstrual cup of claim 1, wherein the plurality of ridges positioned on the exterior surface of the cup are linear.

9. The menstrual cup of claim 1, wherein the plurality of ridges positioned on the exterior surface of the cup are wavy.

10. The menstrual cup of claim 1, wherein the cup comprises at least one hole in the wall of the cup.

11. The menstrual cup of claim 1, wherein:
the cup comprises a lip at the opening,
the lip is an annular protrusion protruding circumferentially around the opening on an interior surface of the cup,
an internal surface of the cup comprises at least one internal rib, and
the at least one internal rib extends from the lip to a base of the cavity.

12. A menstrual cup, comprising:
a cup comprising circumferential walls and an opening, wherein the opening leads to a cavity within the walls;
a stem at a base of the cup, the stem protruding from the cup at a side opposite the opening;
a plurality of ridges positioned on an exterior surface of the cup proximate the base of the cup; and
wherein the cup comprises a lip at the opening, wherein the lip is an annular protrusion protruding circumferentially around the opening on an interior surface of the cup.

13. The menstrual cup of claim 12, wherein the lip comprises a curved surface.

14. The menstrual cup of claim 12, wherein:
the lip comprises an interior curvilinear angled surface and an exterior curvilinear angled surface, and
the interior curvilinear angled surface is convex and the exterior curvilinear angled surface is concave.

15. The menstrual cup of claim 12, wherein the stem protrudes from the cup in a direction orthogonal to a plane of the opening.

16. The menstrual cup of claim 12, wherein the cup comprises at least one hole in the wall of the cup.

17. The menstrual cup of claim 12, wherein the plurality of ridges positioned on the exterior surface of the cup are wavy, and wherein the plurality of ridges are circumferentially equally spaced around the wall of the cup.

18. The menstrual cup of claim 12, wherein the stem comprises grooves circumventing the stem.

19. The menstrual cup of claim 12, wherein the plurality of ridges positioned on the exterior surface of the cup are linear.

20. A menstrual cup, comprising:
a cup comprising a circumferential wall and an opening, wherein the opening leads to a cavity within the wall;
a stem at a base of the cup, the stem protruding from the cup at a side opposite the opening;
a plurality of ridges positioned on an exterior surface of the cup proximate the base of the cup; and
wherein the cup comprises a lip at the opening, wherein the lip is an annular protrusion protruding circumferentially around the opening on an interior surface of the cup, and wherein the lip comprises a curved surface;
wherein the stem protrudes from the cup in a direction orthogonal to a plane of the opening;
wherein the stem comprises grooves circumventing the stem;
wherein the cup comprises at least one hole in the wall of the cup; and
wherein an internal surface of the cup comprises at least one internal rib, and the at least one internal rib extends from the lip to a base of the cavity.
